(19) United States
(12) Patent Application Publication
(10) Pub. No.: US 2004/0121968 A1
(43) Pub. Date: Jun. 24, 2004

(54) Antiangiogenesis by inhibiting protein kinase CK2 activity

(76) Inventors: Alexander Ljubimov, Studio City, CA (US); Raquel Castellon, Norwalk, CA (US); Maria Grant, Fairfield, FL (US)

Correspondence Address: Edward G. Poplawski, Esq., SIDLEY AUSTIN BROWN & WOOD LLP, 40th Floor, 555 West Fifth Street, Los Angeles, CA, 90013-1010, US

(21) Appl. No.: 10/328,646

(22) Filed: Dec. 23, 2002

(51) Int. Cl. A61K 31/7056; A61K 31/12; A61K 35/78

(52) U.S. Cl. 514/43; 514/680; 424/744

(57) ABSTRACT

A method of inhibiting angiogenesis in a mammal is disclosed, which employs a pharmaceutically acceptable composition containing a selective inhibitor of protein kinase CK2 (also known as casein kinase II) enzymatic activity, such as emodin, aloe-emodin, 5,6-dichloro-1-β-D-ribofuranosylbenzimidazole (DRB), and 4,5,6,7-tetrabromobenzotriazole (TBB). Also disclosed is a use of a selective inhibitor of protein kinase CK2 enzymatic activity in the manufacture of a medicament for inhibiting angiogenesis. An in vitro method of screening a potential antiangiogenic agent is also disclosed. A kit for the treatment of a disease by inhibiting angiogenesis is disclosed that contains the pharmaceutically acceptable composition containing a selective inhibitor of protein kinase CK2 enzymatic activity.

The U.S. Government has a paid-up license in this invention and the right in limited circumstances to require the patent owner to license on reasonable terms as provided for by the terms of Grant NIH 1R01 EY 12605, awarded by the National Eye Institute of the National Institutes of Health.

### BACKGROUND OF THE INVENTION

1. Field of the Invention

This invention relates to the medical arts. In particular, it relates to a method for inhibiting angiogenesis.

2. Discussion of the Related Art

Angiogenesis is a highly regulated biological process of sprouting new blood vessels from preexisting blood vessels, which supports growth and maturation. Angiogenesis begins in the mammalian embryo when primitive blood vessels are formed from endothelial precursor cells. Increasingly complex networks of vessels are formed from these primitive precursors. In adults, nonpathogenic angiogenesis is restricted and transient, for example, as part of the wound healing process and during the female reproductive cycle in the endometrium and ovarian follicle.

Because of the role angiogenesis is thought to play in human diseases, pathogenic angiogenesis has been intensively studied. The highly regulated process of angiogenesis is considered a physiological response to the balance between the actions of proangiogenic and antiangiogenic factors, synthesized by endothelial cells, stromal cells, blood, the extracellular matrix, and tumor cells (Carmeliet, P. and Jain, R. K., Angiogenesis in cancer and other diseases, Nature (2000) 407:249-257 [2000]). When proangiogenic factors are synthesized, stimulated by metabolic stress, mechanical stress, inflammation, or genetic mutations, new blood vessels are created from preexisting ones and pathogenic states result (Carmeliet, P. and Jain, R. K. [2000]). Proangiogenic factors create new blood vessels in six distinct steps: vascular destabilization caused by pericyte detachment, extracellular matrix degradation by endothelial proteases, endothelial cell migration, endothelial cell proliferation, tube formation by endothelial cells, and recruitment of pericytes to stabilize vasculature. (Sato, Y., Molecular mechanism of angiogenesis. Transcription factors and their therapeutic relevance, Pharm & Ther 87:51-60 [2000]).

All of these steps are mediated by proangiogenic factors acting in concert with one another. For example, vascular endothelial growth factor (VEGF) and related molecules stimulate vessel leakage, matrix metalloproteases (MMPs) remodel extracellular matrix and release and activate growth factors, platelet-derived growth factor BB (PDGF-BB) and receptors recruit smooth muscle cells, vascular endothelial growth factor receptor (VEGFR) and NRP-1 integrate angiogenic and survival signals, plasminogen activator inhibitor-1 (PAI-1) stabilizes nascent vessels, and angiopoietin 1 (Ang1) and its receptor precursor (Tie2) in turn stabilize vessels (Carmeliet, P. and Jain, R. K. [2000]) 407:249-257).

The survival of tumors is now considered to be dependent upon tumor angiogenesis. For this reason, cancer chemotherapy is beginning to exploit angiogenesis inhibition as a mechanism to limit tumor metastases and angiogenesis is increasingly being used as a diagnostic/prognostic marker. For example, tumor vascularity in solid tumors may inversely correlate with prognosis, and both basic fibroblast growth factor (bFGF; or FGF-2) and VEGF expression have been reported to predict prognosis. (Takahashi, Y. et al., Expression of vascular endothelial growth factor and its receptor, KDR, correlates with vascularity, metastasis, and proliferation of human colon cancer, Cancer Res 55:3964-68 [1995]). Breast cancer prognosis can also be based on the extent of angiogenesis. (Weidner, N. et al, Tumor angiogenesis: a new significant and independent prognostic factor in early-stage breast carcinoma, J. Natl. Cancer Inst. 84:1875-1887 [1992]; Horak, E. R. et al., Angiogenesis, assessed by platelet/endothelial cell adhesion molecule antibodies, as indicator of node metasteses and survival in breast cancer, Lancet 340:1120-1124 [1992]). Not only are tumor growth, progression, and metastasis dependent on access to vasculature, but it is also apparent that an “angiogenic switch” is activated during the transition from mid to late dysplasia, causing a change in tissue angiogenic phenotype preceding the histological tissue transition. (Hanahan, D. and Folkman, J., Patterns and emerging mechanisms of the angiogenic switch during tumorigenesis. Cell. 86:353-64 [1996]).

During tumor-associated angiogenesis, sustained production of angiogenic factors by cancer cells, or indirect macrophage stimulation, causes dysregulated immature vessel growth. (Folkman, J. and Shing, Y., Angiogenesis, J. Biol. Chem. 267:10931-10934[1992]). Several cytokines and growth factors are highly associated with intratumoral angiogenesis, including bFGF and VEGF which modulate angiogenesis in vivo with a paracrine mode of action. (Bikfalvi, A. et al., Biological roles of fibroblast growth factor-2, Endocr. Rev. 18:26-45 [1997]; Ferrara, N. and Davis-Smyth, T., The biology of vascular endothelial growth factor, Endocr Rev 18:4-25 [1997]; Relf, M et al., Expression of the angiogenic factors vascular endothelial cell growth factor, acidic and basic fibroblast growth factor, tumor growth factor-1, platelet-derived endothelial cell growth factor, placenta growth factor, and pleiotrophin in human primary breast cancer and its relation to angiogenesis, Cancer Res. 57(5):963-69 [1997]; Linderholm, B. et al., Vascular endothelial growth factor is of high prognostic value in node-negative breast carcinoma, J. Clin. Oncol. 16:3121-28 [1998]). bFGF and VEGF may synergistically influence angiogenesis, with bFGF modulating endothelial expression of VEGF through both autocrine and paracrine actions. (Seghezzi, G. et al., Fibroblast growth factor-2 (FGF-2) induces vascular endothelial growth factor (VEGF) expression in the endothelial cells of forming capillaries: An autocrine mechanism contributing to angiogenesis, J. Cell. Biol. 141(7):1659-73 [1998]).

For these reasons, drugs acting through an antiangiogenic mechanism are contemplated to prevent neoplastic growth. As an example, Hunter et al described a method of treating a tumor excision site with a composition including paclitaxel or a paclitaxel analog with a polymer to prevent residual blood vessel formation. (U.S. Pat. No. 5,886,026).

In addition to cancer, other pathological states require angiogenesis including diabetes mellitus, Alzheimer's disease, asthma, and hypertension. The pathological progression in endometriosis is also thought to involve angiogensis. (E.g., Taylor, R N et al., Angiogenic factors in endometriosis, Ann N Y Acad Sci 955:89-100 [2002]; Shawki, O et al., Apoptosis and angiogenesis in endometriosis: relationship to development and progression, Fertil Steril. 77 Suppl 1:S44 [2002]; Gazvani, R et al., Peritoneal environment, cytokines and angiogenesis in the pathophysiology of endometriosis, Reproduction 123(2):217-26 [2002]; Taylor, R N et al., Endocrine and paracrine regulation of endometrial angiogenesis, Ann N Y Acad. Sci. 943:109-21 [2001]; Gazvani, R et al., New considerations for the pathogenesis of endometriosis, Int J Gynaecol Obstet. 2002 February; 76(2):117-26 [2002]; Fujimoto, J et al., Angiogenesis in endometriosis and angiogenic factors, Gynecol Obstet Invest. 48 Suppl 1:14-20 [1999]; Healy, D L et al., Angiogenesis: a new theory for endometriosis, Hum. Reprod. Update. 1998 September-October; 4(5):736-40 [1998]; Matsuzaki, S et al., Angiogenesis in endometriosis, Gynecol. Obstet. Invest. 46(2):111-15 [1998]).

Inflammatory disorders can involve excessive angiogenesis in various organs. Blood cells including platelets, mast cells, monocytes, and macrophages release angiogenic factors, such as VEGF, ANG1, bFGF, TGF-β1, PDGF, TNF-α, hepatocyte growth factor (HGF), and insulin-like growth factor (IGF-I). Additionally, blood cells contain proteases that degrade barriers for migrating vasculature and activate growth factors from extracellular matrix. Wound repair is an example of how the inflammatory response influences angiogenesis in a non-pathogenic way. Angiogenesis in wound repair can be described in the following steps: 1) endothelial cells are released from the basement membrane degraded by metalloproteinases and other proteases, and 2) the endothelial cells migrate to connective tissue and differentiate into tubes where they resynthesize the basement membrane, all in response to the proangiogenic factors being secreted at the wound site. (Kleinman, H. K. and Malinda K. M., Role of angiogenesis in wound healing, in Angiogenesis Inhibitors and Stimulators: Potential Therapeutic Implications, Ed. Mousa, S. A., pp.102-109 [2000]).

The primary cause of pathological angiogenesis in non-neoplastic disease states is hypoxia. Hypoxia-induced transcription factors (HIFs) induce the expression of angiogenic factors including VEGF, nitric oxide synthase, PDGF, Ang2, and others (Carmeliet, P. and Jain, R. K. [2000]). As a result, hypoxia-induced angiogenesis leads to blindness in premature newborns, diabetics, and hemorrhagic rupture of atherosclerotic plaques. Additionally, vascular remodeling caused by hypoxia induces chronic obstructive lung disease, characterized by the thickening of vascular muscular coat and pulmonary hypertension. Although hypoxia-induced angiogenesis can be pathological, it also salvages ischemic myocardium and promotes survival after stroke. For these reasons, the use of proangiogenic factors has been proposed as therapy for ischemic diseases, such as arteriosclerotic occlusion of the lower limb or angina pectoris/myocardial infarction.

Diabetic retinopathy, the most severe ocular complication of diabetes mellitus, may be defined as a disease of retinal microvasculature. Diabetic retinopathy is the leading cause of new blindness in persons 25 to 74 years of age in the United States, accounting for about 8,000 new blindness cases each year. (Aiello L P et al., Diabetic retinopathy, Diabetes Care 21:143-156 [1998]; Lim J I et al., Review of diabetic retinopathy, Curr. Opin. Ophthalmol. 2:315-323 [1991]). Two types of diabetic retinopathy are recognized clinically: (1) nonproliferative diabetic retinopathy (NPDR), associated with retinal ischemia, pericyte loss, capillary closure, retinal infarctions/cotton wool spots, retinal hemorrhages, microaneurisms, intraretinal microvascular abnormalities, and macular edema; and (2) proliferative diabetic retinopathy (PDR), associated with intravitreal hemorrhages, optic disc or peripheral neovascularization, preretinal fibrovascular membranes, and vitreoretinal traction with retinal detachments (Aiello L P et al. [1998]; Lim J I et al. [1991]). Sadly, 43% of juvenile-onset and 60% of adult-onset diabetics lose vision within 5 years of the onset of PDR.

Supporting the conclusion that diabetic retinopathy is a disease of retinal microvasculature, abnormally high concentrations of angiogenic growth factors have been detected in the vitreous of diabetic retinopathy and PDR patients. (Aiello L P, and Hata Y., Molecular mechanisms of growth factor action in diabetic retinopathy, Curr. Opin. Endocrinol. Diabetes 6:146-156 [1999]; Boulton, M. et al., Intravitreal growth factors in proliferative diabetic retinopathy: correlation with neovascular activity and glycaemic management, Br. J. Ophthalmol. 81:228-233 [1997]; Freyberger, H. et al., Increased levels of platelet-derived growth factor in vitreous fluid of patients with proliferative diabetic retinopathy, Exp. Clin. Endocrinol. Diabetes 108:106-109 [2000]). Additionally, VEGF induced by hypoxia and hyperglycemia has been implicated in causing PDR neovascularization and vascular hyperpermeability. (Aiello L P, and Hata Y., Molecular mechanisms of growth factor action in diabetic retinopathy, Curr. Opin. Endocrinol. Diabetes 6:146-156 [1999]; Aiello, L P and Wong, J S, Role of vascular endothelial growth factor in diabetic vascular complications, Kidney Int. 58 (Suppl. 77): 113-119 [2000]).

Retinas in proliferative diabetic retinopathy (PDR) have increased expression of VEGF, PIGF, and tenascin, a vascular basement membrane protein. (E.g., Ljubimov A V el al., Basement membrane abnormalities in human eyes with diabetic retinopathy, J. Histochem. Cytochem. 1996;44:1469-1479 [1996]; Spirin K S et al., Basement membrane and growth factor gene expression in normal and diabetic human retinas, Curr. Eye Res. 18:490-499 [1999]). Hypoxia-inducible VEGF is considered as the main growth factor that mediates PDR neovascularization (Smith L E et al., Regulation of vascular endothelial growth factor-dependent retinal neovascularization by insulin-like growth factor-1 receptor, Nat. Med. 5:1390-1395 [1999]).

However, VEGF inhibitors only partially prevent ocular neovascularization and vessel hyperpermeability. (Campochiaro, P A, Retinal and choroidal neovascularization, J. Cell Physiol. 184:301-310 [2000]; Aiello L P, Vascular endothelial growth factor. 20th-century mechanisms, 21st-century therapies. Invest. Ophthalmol. Vis. Sci. 38:1647-1652 [1997]; Ozaki H et al., Blockade of vascular endothelial cell growth factor receptor signaling is sufficient to completely prevent retinal neovascularization, Am. J. Pathol. 156:697-707 [2000]; Aiello L P, Vascular endothelial growth factor and the eye: Biochemical mechanisms of action and implications for novel therapies, Ophthalmic Res. 1997;29:354-362; Aiello L P et al., Vascular endothelial growth factor-induced retinal permeability is mediated by protein kinase C in vivo and suppressed by an orally effective β-isoform-selective inhibitor, Diabetes 46:1473-1480 [1997]; Campochiaro P A, Retinal and choroidal neovascularization, J. Cell Physiol. 184:301-310 [2000]; Penn J S, Bullard L E, VEGF signal transduction proteins ERK-1 and ERK-2 are targets for the inhibition of retinal angiogenesis, Exp. Eye Res. (ICER Abstracts) 71(Suppl. 1):S.5 [2000]).

This implies that other factors may be involved in this process. Growth factor synergies have been reported in other tissues. (Goto F et al., Synergistic effects of vascular endothelial growthfactor and basic fibroblast growth factor on the proliferation and cord formation of bovine capillary endothelial cells within collagen gels, Lab. Invest. 69:508-517 [1993]; Stavri G T et al., Hypoxia andplatelet-derived growth factor-BB synergistically upregulate the expression of vascular endothelial growth factor in vascular smooth muscle cells, FEBS Lett. 358:311-315 [1995a]; Stavri G T el al., Basic fibroblast growth factor upregulates the expression of vascular endothelial growth factor in vascular smooth muscle cells. Synergistic interaction with hypoxia, Circulation 92:11-14 [1995b]; Hata Y et al., Basic fibroblast growth factor induces expression of VEGF receptor KDR through a protein kinase C and p44/p42 mitogen-activated protein kinase-dependent pathway, Diabetes 48:1145-1155 [1999]; Miele C et al., Insulin and insulin-like growth factor-I induce vascular endothelial growth factor mRNA expression via different signaling pathways, J. Biol. Chem. 275:21695-21702 [2000]).

Protein kinase CK2 (formerly known as casein kinase II) is a serine/threonine kinase implicated in cell replication, cellular survival, and tumorigenesis via a role in protooncogene Wnt-1-mediated signaling. (E.g., Song, D. et al., Endogenous protein kinase CK2 participates in Wnt signaling in mammary epithelial cells, J. Biol. Chem. 275(31):23790-97 [2000]). Kim et al. taught a composition comprising thrombin, protein kinase CK2, and sphingosine or a sphingosine derivative, for treating patients with hemophilia, ulcers, or other microbial infections, in addition to reducing clotting time during blood vessel suturing. (Kim et al., U.S. Pat. No. 5,897,860). But a role for protein kinase CK2 in angiogenesis was heretofore unknown.

There remains a need for a method of inhibiting angiogenesis, in vivo. As an example, angiogenesis inhibition is desired to inhibit vascularization of solid malignant tumors or to inhibit the development of retinopathies or endometriosis. In vitro screening for potential new antiangiogenic agents is facilitated by useful positive controls. These and other benefits are provided by the present invention as described herein.

### SUMMARY OF INVENTION

The invention described herein relates to a method of inhibiting angiogenesis in a mammal by inhibiting the activity of protein kinase CK2 (also herein “CK2” or “CKII”). The method involves administering to the mammal a pharmaceutically acceptable composition comprising a selective inhibitor of protein kinase CK2 enzymatic activity, such that an effective amount of the inhibitor is delivered to a tissue in the mammal. The tissue in the mammal comprises endothelial cells. By virtue of practicing the method, protein kinase CK2 enzymatic activity is inhibited in a plurality of the cells, thereby resulting in an antiangiogenic effect in the tissue. Benefits of the present inventive method include the treatment of malignant tumors or various proliferative retinopathies or endometriosis, by inhibiting the development of neomicrovasculature.

The present invention is also directed to the use of an inhibitor of protein kinase CK2 enzymatic activity in the manufacture of a medicament for inhibiting angiogenesis. The medicament comprises a pharmaceutically acceptable composition comprising the inhibitor of protein kinase CK2 enzymatic activity.

The present invention also relates to an in vitro method of screening a potential antiangiogenic agent. The in vitro method involves using the CK2 inhibitor as a positive control for detecting antiangiogenic properties of potential new antiangiogenic agents. In accordance with the in vitro method, a plurality of mammalian endothelial cells is cultured in the presence of signal molecules, such as but not limited to, vascular endothelial growth factor (VEGF), placenta growth factor (PlGF), insulin-like growth factor (IGF)-I, platelet-derived growth factor (PDGF)-BB, and/or fibroblast growth factor (FGF)-2, that induce proliferation, survival, migration, and/or sprouting of the cells; then a first population of the plurality of mammalian endothelial cells is exposed to the potential antiangiogenic agent, and any detectable effect of the agent on cellular proliferation, survival, migration, and/or sprouting in the first population is determined. Further, separately from the first population, a second population of the plurality of mammalian endothelial cells is exposed to a selective inhibitor of protein kinase CK2 enzymatic activity, in an amount sufficient to inhibit proliferation, survival, migration, and/or sprouting of the endothelial cells in the presence of the signal molecules, and detecting an inhibitory effect on cellular proliferation, survival, migration, and/or sprouting in the second population. The detected effect of the potential antiangiogenic agent on cellular proliferation, survival, migration, and/or sprouting in the first population is compared with the detected inhibitory effect of the selective inhibitor of protein kinase CK2 enzymatic activity on cellular proliferation, survival, migration, and/or sprouting in the second population. If an inhibitory effect in the first population is similar to the inhibitory effect in the second population, this indicates an antiangiogenic property of the potential antiangiogenic agent. Once the antiangiogenic potential of a chemical agent is identified by the in vitro method, then further research can be done to further purify the active component of the substance (e.g., if the substance is a mixture, not a compound), verify its actual effect in vivo, and ascertain its clinical usefulness. Thus, the inventive in vitro method facilitates the screening and development of new pharmaceuticals for the treatment of cancer and other diseases, in which inhibiting the formation of neomicrovasculature is a likely therapeutic target.

Useful kits are also provided for facilitating the practice of the inventive methods.

These and other advantages and features of the present invention will be described more fully by way of the drawings and in a detailed description of the preferred embodiments which follows. By way of further describing the present invention, the disclosure and drawings of commonly owned U.S. patent application Ser. No. ______, simultaneously filed on ______, 2002, and entitled SECONDARY SPROUTING FOR ISOLATION AND EXPANSION OF ENDOTHELIAL SPROUT CELLS AND ENDOTHELIAL PRECURSOR CELLS FROM A MIXED POPULATION AND FOR SCREENING SUBSTANCES, are incorporated herein by reference in their entirety.

### BRIEF DESCRIPTION OF THE DRAWINGS

FIG. 1 shows synergistic growth factor-mediated increase of retinal endothelial cell (REC) proliferation. Bovine REC were treated for 6 days with-10 ng/mL each of the indicated growth factors (GFs) in medium with 0.5% serum. Cell numbers were measured with MTS assay. Note a dramatic threefold increase of cell number after treatment with a combination VEGF+IGF-I+FGF-2+PlGF (“Four GFs”) compared to single or paired growth factors. Bars are mean±SEM of at 3-7 experiments in triplicate. *, p<0.05 vs. control.

FIG. 2 shows secondary sprouting on BD Matrigel™ (a basement membrane matrix). REC form capillary-like tubes (FIG. 2A, FIG. 2B). In 24 hr, tubes start shortening (FIG. 2C), cells aggregate into clumps (FIG. 2D), and reportedly die by apoptosis (i.e., programmed cell death) within 48 hr. (Albini A, Tumor and endothelial cell invasion of basement membranes. The Matrigel chemoinvasion assay as a tool for dissecting molecular mechanisms, Pathol. Oncol. Res. 4:230-241 [1998]). Due to a longer examination time, it was unexpectedly observed that the supposedly dead aggregates contained living cells that by day 5 proliferated, migrated and invaded basement membrane matrix (BD Matrigel™) forming three-dimensional spheres (FIG. 2E). In some instances, separate spheres initiated cell-cell contacts resulting in connecting structures resembling larger capillaries (FIG. 2F). This secondary sprouting process was greatly enhanced by the addition of 10 ng/mL PDGF-BB (FIG. 2G) or FGF-2 (FIG. 2H). Pictures were taken in a Leica inverted microscope with a 4×(FIGS. 2A-2E, 2G, and 2H) or a 10×(FIG. 2F) objective. (See, Castellon, R. et al., Effects of Angiogenic Growth Factor Combinations on Retinal Endothelial Cells, Exp. Eye Res. 74:523-35 [2002]).

FIG. 3 shows significant inhibitory effect of specific CK2 inhibitors on growth factor (GF)-mediated cell migration. Confluent bovine REC monolayers were wounded and cultured for 7 days in 0.5% serum-containing medium with four growth factors (IGF-1+FGF-2+VEGF+PlGF at 10 ng/ml each)±CK2 inhibitors, emodin (10 μM) or DRB (15 μM). Cell migration into the wound was counted using the AAB software. Bars represent mean±SEM of at least 3 individual experiments. *, p values of CK2 inhibitor vs. four GFs.

FIG. 4 shows the effect of the CK2 inhibitor DRB on bovine REC proliferation and survival. Cells were plated in medium with 0.5% (survival) or 10% serum (proliferation) containing various concentrations of DRB. The number of live cells was measured on day 6 with MTS assay. Bars represent mean±SDEM of two individual experiments in triplicate. *=p<0.05.

FIG. 5 shows the effect of the CK2 inhibitor DRB on bovine REC secondary sprouting. Cells were seeded on Matrigel™ in medium with 0.5% serum containing various concentrations of DRB. The number of live cells was measured on day 9 with MTS assay. Bars represent mean±SDEM of two individual experiments in duplicate. *=p<0.05.

FIG. 6 shows representative fluorescein angiograms of the retina from a vehicle-treated control mouse (FIG. 6A) and of the retina from an emodin-treated mouse (FIG. 6B). Arrows show neovascular tufts prominent in the vehicle-treated animals.

FIG. 7 shows a quantitation of preretinal neovascularization in untreated, vehicle-treated and emodin-treated mouse retinas.

FIG. 8 shows a quantitation of preretinal neovascularization in untreated, vehicle-treated and DRB-treated mouse retinas.

FIG. 9 shows CK2 α subunit expression in cultured REC of normal (N) and diabetic retinopathic (DR) origin as detected by immunohistochemistry. These immunofluorescent pictures were taken with the same exposure time.

### DETAILED DESCRIPTION OF THE PREFERRED EMBODIMENTS

The inventive method of inhibiting angiogenesis in a mammal includes administering to the mammal an inhibitor of protein kinase CK2 enzymatic activity. The method is useful for producing an antiangiogenic effect in any mammal, including a human, non-human primate, canine, feline, bovine, porcine or ovine mammal, as well as in a small mammal such as a rodent (e.g., mouse, rat, gerbil, hamster, guinea pig) or lagomorph (e.g., rabbit).

An “antiangiogenic effect” is an inhibition of one or more processes involved in angiogenesis, including in vivo, the dissolution of extracellular matrix (e.g., invasion) and the growth and survival of cells forming new blood vessels (e.g., endothelial cells, endothelial precursor cells, and pericytes), and as detectable in vitro, the inhibition of endothelial cell proliferation, survival, migration, and/or sprouting.

In accordance with the inventive method, the CK2 inhibitor is delivered to a tissue of the mammal that contains vascular endothelial cells capable of forming vascular structures in response to an appropriate combination of signal molecules. A “tissue” is a group of similar cells united to perform a specific physiologic function. The tissue can be organized as an organ, for example, an eye, a kidney, or skin, or as a subpart of an organ, such as retinal tissue or endometrial tissue. A tissue can also be a solid tumor, e.g., a malignant tumor, such as but not limited to, a glioma, a glioblastoma, an oligodendroglioma, an astrocytoma, an ependymoma, a primitive neuroectodermal tumor, an a typical meningioma, a malignant meningioma, a neuroblastoma, a sarcoma, a melanoma, a lymphoma, or a carcinoma. The malignant tumor tissue can be contained within any structure of the mammal, including the skull, brain, spine, thorax, lung, abdomen, peritoneum, prostate, ovary, uterus, breast, stomach, liver, bowel, colon, rectum, bone, lymphatic system, eye, ear, or skin, of the mammalian subject.

Protein kinase CK2 (“CK2” or “CKII”; EC 2.7.1.37) is also known as “casein kinase II”. (See, e.g., Niefind, K. et al., Crystal Structure of Human Protein Kinase CK2: Insights Into Basic Properties of the CK2 Holoenzyme, EMBO J. 20 pp. 5320 [2001]).

The inhibitor of protein kinase CK2 is a substance, such as a compound, the selective binding of which, in vivo or in vitro, to a site on CK2 results in a reduction of CK2 enzymatic activity, compared to an appropriate control that lacks the substance. In one embodiment, the inhibitor of protein kinase CK2 (“CK2 inhibitor”) is 5,6-dichloro-1-β-D-ribofuranosylbenzimidazole (“DRB”). Alternatively, the CK2 inhibitor is emodin (3-methyl-1,6,8-trihydroxyanthraquinone or 6-methyl-1,3,8-trihydroxyanthraquinone; Beilstein Registry Number: 1888141). Another embodiment is aloe-emodin (1,8-dihydroxy-3-hydroxymethylanthraquinone). Another embodiment of the CK2 inhibitor is 4,5,6,7-tetrabromobenzotriazole (i.e., 4,5,6,7-tetrabromo-2-azabenzimidazole; “TBB”; e.g., Sarno, S et al., FEBS Lett 496(1):44-48 [2001]; Battistutta, R et al., Protein Sci. 10(11):2200-06 [2001]). Also included among useful CK2 inhibitors are pharmaceutically acceptable molecular conjugates or salt forms of emodin, aloe-emodin, DRB, or TBB, that still have activity as CK2 inhibitors as defined herein. Examples of pharmaceutically acceptable salts of CK2 inhibitors, include sulfate, chloride, carbonate, bicarbonate, nitrate, gluconate, fumarate, maleate, or succinate salts. Other embodiments of pharmaceutically acceptable salts contain cations, such as sodium, potassium, magnesium, calcium, ammonium, or the like. Other embodiments of useful CK2 inhibitors are hydrochloride salts. For providing enhanced cell permeability to a CK2 inhibitor moiety, various conjugated forms are useful, e.g., CK2 inhibitor-lipid conjugates, emulsified conjugates of CK2 inhibitors, lipophillic conjugates of CK2 inhibitors, and liposome- or micelle-conjugated CK2 inhibitors. (Fenske, D B et al., Cationic poly(ethyleneglycol) lipids incorporated into pre-formed vesicles enhance binding and uptake to BHK cells, Biochim Biophys Acta 1512(2):259-72 [2001]; Khopade, A J et al., Concanavalin-A conjugated fine-multiple emulsion loaded with 6-mercaptopurine, Drug Deliv. 2000 April-June; 7(2):105-12 [2000]; Lambert, D M et al., Rationale and applications of lipids as prodrug carriers, Eur. J. Pharm. Sci. 11 Suppl 2:S15-27 [2000]; Pignatello, R et al., Lipophilic methotrexate conjugates with antitumor activity, Eur J Pharm Sci. 10(3):237-45 [2000]; Allen, C et al., PCL-b-PEO micelles as a delivery vehicle for FK506: assessment of a functional recovery of crushed peripheral nerve, Drug Deliv. 7(3):139-45 [2000]; Dass, C R et al., Liposomes containing cationic dimethyl dioctadecyl ammonium bromide: formulation, quality control, and lipofection efficiency, Drug Deliv. 9(1):11-8 [2002]; Dass, C R, Apolipoprotein A-I, phospholipid vesicles, and cyclodextrins as potential anti-atherosclerotic drugs: delivery, pharmacokinetics, and efficacy, Drug Deliv. 7(3):161-82 [2000]).

Although the practice of the inventive methods does not require the measurement of CK2 enzymatic activity, enzymatic assay methods for determining CK2 activity are known in the art, and thus inhibition of CK2 enzymatic activity can be detected. (E.g., Dobrowolska G et al., CK2, a protein kinase of the next millennium, Mol. Cell. Biochem. 191:3-12 [1999]; Sayed M et al., Stress-induced activation of protein kinase CK2 by direct interaction with p38 mitogen-activated protein kinase, J. Biol. Chem. 275:16569-16573129 [2000]). For example, a typical CK2 activity assay involves: (1) lysate preparation. Cells are typically washed with ice-cold PBS, scraped and lysed in 50 mM HEPES, pH 7.2, containing 100 mM NaCl, 1 MM EGTA and 20 MM NaF in buffer A (1 mM sodium orthovanadate, 1% aprotinin, 1 mM PMSF, 1 μM pepstatin, 10 μg/mL soybean trypsin inhibitor, 0.5 μg/ml leupeptin, and 1% NP-40). Lysates are produced by brief sonication and are microcentrifuged at 15,000 rpm for 5 min. Supernatant fractions are collected and can be stored frozen. Protein can be determined with BCA assay (Pierce) or any other conventional protein assay. (2) CK2 activity assay. Typically, triplicate aliquots of cell lysates are incubated in a final volume of about 25 μL with 0.5 mM CK2-specific substrate peptide RRRADDSDDDDD (SEQ ID NO: 1; Calbiochem) and 100 μM [³²P]-ATP (10 μCi/assay) in 12 mM MOPS, pH 7.2, and 15 mM MgCL₂ for 15 min at 30° C. At the end of incubation, the mixture is spotted, e.g., on a 1.5 cm² piece of Whatman P-81 paper, the filter is washed in 1% phosphoric acid, transferred to scintillation vials with 0.5 ml scintillation fluid and the incorporated radioactivity is measured in a scintillation counter (e.g., Beckman Instruments). Specific radioactivity is determined by subtracting negative control counts in the presence of DRB, TBB, or emodin (specific CK2 inhibitors), from total counts without DRB, TBB, or emodin. Calibration curve with purified enzyme is made the same way using 0.05-2 mU of purified CK2 holoenzyme (Calbiochem).

The CK2 inhibitor can be synthesized by known chemical means or can be procured commercially (e.g., Sigma-Aldrich). Emodin and aloe-emodin are also typically isolated from the root and rhizomes of Rheum palmatum (Polygonaceae) or from the leaves of Aloe vera, respectively, and can be purified by known means. (E.g., Mueller, S. et al., Biotransformation of the anthraquinones emodin and chrysophanol by cytochrome P450 enzymes, Drug Metabolism and Disposition 26(6):540-46 [1998]; Pecere, T et al., Aloe-emodin is a new type of anticancer agent with selective activity against neuroectodermal tumors, Cancer Res. 60:2800-04 [2000]). Emodin can also be isolated and purified from Ventilago leiocarpa Bunge (Rhamnaceae), Rhamnus triquerta, Polygonum multiflorum, Polygonum cuspidatum, and Artemisia scoparia. (E.g., Lin, C C et al., Hepatoprotective effects of emodin from Ventilago leiocarpa, J. Ethnopharmacol. 52(2):107-11 [1996]; Jayasuriya, H et al., Emodin, a protein tyrosine kinase inhibitor from Polygonum cuspidatum, J. Nat. Prod. 55(5):696-98 [1992]; Huang, H C et al., Vasorelaxants from Chinese herbs, emodin and scaparone, possess immunosuppressive properties, Eur. J. Pharmacol. 198(2-3):211-213 [1991]; Goel, R K et al., Antiulcerogenic and anti-inflammatory effects of emodin, isolatedfrom Rhamnus triquerta wall, Indian J. Exp. Biol. 29(3):230-32 [1991]). Aloe-emodin can also be isolated and purified from the leaves of Picramnia antidesma spp. (Solis, P N et al., Bioactive anthraquinone glycosides from Picramnia antidesma spp., Phytochemistry 38(2):477-80 [1995]).

In accordance with the present invention, the pharmaceutically acceptable composition contains the CK2 inhibitor and, optionally, contains pharmaceutically acceptable solvent(s), adjuvant(s) and/or pharmaceutically acceptable non-medicinal, non-toxic carrier(s), binder(s), thickener(s), and/or filler substance(s) that are known to the skilled artisan for the formulation of tablets, pellets, capsules, solutions, emulsions, suspensions, and any other form suitable for use. The carriers which can be used include glucose, lactose, sucrose, gum acacia, gelatin, mannitol, starch, starch paste, magnesium trisilicate, talc, corn starch, keratin, colloidal silica, potato starch, urea, medium chain length triglycerides, dextrans, petrolatum, and other carriers suitable for use in manufacturing preparations, in solid, semisolid, or liquid form. In addition auxiliary, stabilizing, thickening and coloring agents and perfumes can be used. Also contemplated are additional medicinal or nutritive additives in combination with at least one CK2 inhibitor, as may be desired to suit the more particular needs of the practitioner.

Also useful as optional components of the pharmaceutically acceptable composition, are additional medicinal or nutritive additives, as may be desired to suit the more particular needs of the practitioner. Examples of optional nutritive additives include vitamins, such as vitamin A, C, or E. An example of an optional medicinal additive, especially useful in topical applications, is one or more antibiotic, such as ciprofloxacin, penicillin, fluoroquinolone, erythromycin, rifampicin, bacitracin, or streptomycin, in conventional amounts. Other embodiments useful used in combination therapies for cancers, can optionally contain therapeutic cytotoxic agents (e.g., cisplatin, carboplatin, methotrexate, 5-fluorouracil, amphotericin), commonly used to treat malignancies. Other embodiments include combination therapies employing the pharmaceutically acceptable composition containing the CK2 inhibitor together with one or more angiostatic steroids (e.g., 2-methoxy-estradiol), angiogenic growth factor antagonists (e.g., soluble receptors, R & D Chimeras Systems), integrin antagonists, RGD-containing proteins and peptides, natural antiangiogenic proteins (e.g., platelet factor 4, angiostatin, endostatin, thrombospondins, pigment epithelium-derived factor [PEDF]), somatostatin analogs, such as octreotide (e.g., sandostatin [Novartis]), and/or antagonists of protein kinase C-β. Such chemotherapeutic agents can be combined with the CK2 inhibitor as a constituent of the pharmaceutically acceptable composition, or they can be administered separately but in conjunction with the inventive method. An advantage of the present inventive method is that lower effective doses of cytotoxic or other chemotherapeutic agents can be given to a patient when used in conjunction with a selective CK2 inhibitor, with lower toxic risk to the patient and better quality of life. However, the skilled practitioner will still carefully monitor the patient for symptoms of general toxicity from the anti-cancer treatment, such as blurred vision, nausea, fever, elevated hepatic enzymes, inflammation, non-tumor necrosis, hemorrhage, bloody stool, and/or hair loss.

The pharmaceutically acceptable composition containing the CK2 inhibitor is administered by any suitable method. Representative methods include giving, providing, feeding, dispensing, inserting, injecting, infusing, perfusing, prescribing, furnishing, treating with, taking, ingesting, swallowing, eating, inhaling, spraying, spreading, attaching or applying a pharmaceutically acceptable composition containing the CK2 inhibitor. Methods of administering are well known to those of skill in the art and include most preferably parenteral administration, oral administration, and/or enteral administration.

In one preferred embodiment, administration of the pharmaceutically acceptable composition is local, for example, by intravitreous injection, stereotactic injection, or by topical application, for example, to the skin, genital tissues, or cornea. For such topical uses the pharmaceutically acceptable composition can be formulated in any suitable way, e.g., as an injectable liquid (e.g., an aqueous solution or suspension in normal saline or PBS), or in the form of a patch, cream, gel, ointment, spray, or eye drops.

Another preferred embodiment of the present method involves administration by a systemic delivery route, i.e., a route whereby CK2 inhibitor is delivered to a tissue primarily via the blood stream. Entry of CK2 inhibitors into the blood stream of a human can occur by any route, system, device, or medium. Thus, in some embodiments the useful pharmaceutically acceptable composition is formulated as an inhaler or intranasal spray. In another embodiment, the useful pharmaceutically acceptable composition is formulated for parenteral administration as an injectable liquid (e.g., an aqueous solution or suspension in normal saline or PBS, or lipid-containing carrier). In some useful embodiments, the systemic delivery route is by intramuscular or subcutaneous injection. Some other useful systemic delivery systems involve a transvascular delivery route, for example, by intravenous or intra-arterial injection or infusion. For treating an intracranial tumor, the CK2 inhibitor is administered to the mammalian subject, for example, by intracarotid infusion or intracranial pump with or without catheter.

For the purposes of the present invention, a systemic delivery route can also include an ingestive delivery route, or a parenteral delivery route, for example, a transdermal or transmucosal delivery route. Transmucosal delivery routes include delivery of the CK2 inhibitor through the mucosa or epithelium of the mouth including the sublingual epithelium, through the vaginal epithelium, or through the rectal epithelium.

Other useful systemic delivery systems are known and include, but are not limited to, implant; transmucosal delivery matrices; or suppositories or gels.

Another embodiment of the useful pharmaceutically acceptable composition of the present invention is a formulation for systemic transmucosal delivery of at least one CK2 inhibitor. A variety of pharmaceutically acceptable systems for transmucosal delivery of therapeutic agents are known in the art and are compatible with the practice of the present invention. (Heiber et al., Transmucosal delivery of macromolecular drugs, U.S. Pat. Nos. 5,346,701 and 5,516,523; Longenecker et al., Transmembrane formulations for drug administration, U.S. Pat. No. 4,994,439). Transmucosal delivery devices may be in free form, such as a cream, gel, or ointment, or may comprise a determinate form such as a tablet, patch, or troche. For example, delivery of at least one CK2 inhibitor may be via a transmucosal delivery system comprising a laminated composite of, for example, an adhesive layer, a backing layer, a permeable membrane defining a reservoir containing at least one CK2 inhibitor, a peel seal disc underlying the membrane, one or more heat seals, and a removable release liner. (Ebert et al., Transdermal delivery system with adhesive overlay and peel seal disc, U.S. Pat. No. 5,662,925; Chang et al., Device for administering an active agent to the skin or mucosa, U.S. Pat. Nos. 4,849,224 and 4,983,395).

Alternatively, a tablet or patch for delivery through the oral mucosa can comprise an inner layer containing the therapeutic agent of choice, a permeation enhancer, such as a bile salt or fusidate, and a hydrophilic polymer, such as hydroxypropyl cellulose, hydroxypropyl methylcellulose, hydroxyethylcellulose, dextran, pectin, polyvinyl pyrrolidone, starch, gelatin, or any of a number of other polymers known to be useful for this purpose. This inner layer can have one surface adapted to contact and adhere to the moist mucosal tissue of the oral cavity and may have an opposing surface adhering to an overlying non-adhesive inert layer. Optionally, such a transmucosal delivery system can be in the form of a bilayer tablet, in which the inner layer also contains additional binding agents, flavoring agents, or fillers. Some useful systems employ a non-ionic detergent along with a permeation enhancer. These examples are merely illustrative of available transmucosal delivery technology and are not limiting of the present invention.

Another embodiment of the pharmaceutically acceptable composition is a gel for systemic delivery of at least one CK2 inhibitor via the rectal or vaginal mucosa, similar to gels commonly used for the delivery of various other therapeutic agents. Hydrogel matrices are known for this purpose. (e.g., Feijen, Biodegradable hydrogel matrices for the controlled release of pharmacologically active agents, U.S. Pat. No. 4,925,677). Such biodegradable gel matrices can be formed, for example, by cross-linking a proteinaceous component and a polysaccharide or mucopolysaccharide component, then loading with at least one CK2 inhibitor to be delivered. Other conventional rectal or intravaginal suppository systems are also usefully employed for delivering CK2 inhibitors in accordance with the invention.

Another embodiment of the pharmaceutically acceptable composition of the present invention is one formulated for the systemic delivery of at least one CK2 inhibitor via a biodegradable matrix or osmotic pump implanted within the body or under the skin of a human or non-human vertebrate. The implant matrix may be a hydrogel similar to those described above. Alternatively, it may be formed from a poly-alpha-amino acid component. (Sidman, Biodegradable, implantable drug delivery device, and process for preparing and using same, U.S. Pat. No. 4,351,337).

The pharmaceutically acceptable compositions can be formulated for oral or enteral administration, for example, as tablets, troches, caplets, microspheres, hard or soft capsules, lozenges, aqueous or oily suspensions, dispersible powders or granules, emulsions, syrups, elixirs or enteral formulas.

Compositions intended for oral use are prepared according to any method known to the art for the manufacture of pharmaceutical compositions. Compositions can also be coated by the techniques described in the U.S. Pat. Nos. 4,256,108; 4,160,452; and 4,265,874, to form osmotic therapeutic tablets for controlled release. Other techniques for controlled release compositions, such as those described in the U.S. Pat. Nos. 4,193,985; and 4,690,822; 4,572,833 can be used in the formulation of the inventive pharmaceutically acceptable compositions.

Controlled release or continuous dosing regimens are also useful. The pharmaceutical industry has developed all sorts of slow and/or sustained-release technology. Sustained-release formulations employ several methods. The most common is a tablet containing an insoluble core; a drug applied to the outside layer is released soon after the medication is ingested, but drug trapped inside the core is released more slowly. Capsules containing multiparticulate units of drug with coatings that dissolve at different rates are designed to give a sustained-release effect.

In accordance with the invention, pharmaceutically acceptable compositions are formulated to deliver an effective dose of at least one CK2 inhibitor by the above-described or any other pharmaceutically acceptable systemic delivery system, preferably in an amount of about 10 to about 100 milligrams per kilogram of body mass per dose of CK2 inhibitor, more preferably about 20 to about 80 milligrams per kilogram of body mass per dose, and most preferably about 25 to about 50 milligrams per kilogram of body mass per dose. Preferably, one to two doses of the CK2 inhibitor are delivered to the mammal each day, more preferably two to four doses of the CK2 inhibitor are delivered daily, although more than four daily doses are also in accordance with the present invention. The useful pharmaceutically acceptable composition can be formulated and manufactured at more than one concentration unit of CK2 inhibitor, such that modular incremental amounts of CK2 inhibitors are easily administered to subjects of various sizes as needed.

In other embodiments, administration of the CK2 inhibitor to the mammalian subject, for delivery to, e.g., a malignant tumor, is by intratumoral injection through a surgical incision, for example, through a craniotomy for a brain tumor. Typically, but not necessarily, surgical debulking of the tumor is done, if possible, before injection of the CK2 inhibitor into the remaining tumor mass containing malignant cells. Also for treating a malignant tumor, another preferred delivery method is stereotactic injection of the CK2 inhibitor into the malignant tumor at a site having pre-established coordinates, e.g., in the brain, or sustained release by an implanted osmotic pump.

In accordance with the inventive method, administration by injection can be in a bolus or by infusion over a period of one to thirty minutes, and most preferably during a period of one to about fifteen minutes. If by infusion, the practitioner skilled in the art is also cautious in regulating the total infusion volume, rate of liquid infusion, and electrolyte balance to avoid adverse physiological effects related to these.

For example, for delivery by intravascular infusion or bolus injection into a mammal, such as a human, the CK2 inhibitor is preferably in a solution that is suitably balanced, osmotically (e.g., about 0.15 M saline) and with respect to pH, typically between pH 7.2 and 7.5; preferably the solution further comprises a buffer, such as a phosphate buffer (e.g., in a phosphate buffered saline solution). The solution is formulated to deliver a dose of about 10 to 100 milligrams of CK2 inhibitor per kilogram body mass in a pharmaceutically acceptable fluid volume over a maximum of about thirty minutes.

In accordance with the inventive method, administration of the pharmaceutically acceptable composition containing the CK2 inhibitor is preferably, but not necessarily, repeated, as described herein above, for a series of treatments lasting over about five to about 10 consecutive days. With careful clinical monitoring, multiple series of treatments with intervening non-treatment periods (e.g., about one month) can be applied, as needed and to avoid toxicity to an individual patient.

Some useful CK2 inhibitors, such as emodin or DRB, are not easily dissolved in water; in preparing these agents for administration, a suitable and pharmaceutically acceptable solvent, such as ethanol (e.g., 25% v/v ethanol or higher ethanol concentrations), can be used to dissolve the CK2 inhibitor, prior to further dilution with an infusion buffer, such as PBS. The skilled practitioner is cautious in regulating the final concentration of solvent in the infusion solution to avoid solvent-related toxicity. For example, a final ethanol concentration in an infusion solution up to 5-10% (v/v) is tolerated by most mammalian subjects with negligible toxicity.

Alternatively, in some embodiments, phosphate-buffered saline with 20% polyethylene glycol 400 (PEG 400)+2% Tween-80, pH 7.2, can be used as a vehicle for mixing emodin, DRB, or TBB for delivery. The final mixture is a suspension that is sonicated or vortexed briefly before intraperitoneal, intramuscular, or intravitreal injection, but which is not useful for transvascular (i.e., intravenous or intraarterial) delivery.

Alternatively, about 50-100% v/v dimethyl sulfoxide (DMSO; aqueous, if less than 100%) can be used to solubilize the CK2 inhibitor, although the odor of DMSO can be unpleasant to some mammals and can lead to aggressive or violent behaviors by cagemates toward DMSO-treated animals.

In one embodiment directed to inhibiting angiogenesis in a brain tumor, the CK2 inhibitor is injected directly into the tumor, most preferably by stereotactic injection means known in the art. Alternatively, injection with CK2 inhibitor can be by intraarterial (e.g., intracarotid) or intravenous injection or infusion, in conjunction with at least transient disruption of the blood brain barrier by physical or chemical means, delivered simultaneously with the CK2 inhibitor.

“Simultaneously” means that the physical or chemical means for disrupting the blood brain barrier are administered contemporaneously or concurrently with the CK2 inhibitor. “Simultaneously” also encompasses disrupting means being administered within about one hour after the CK2 inhibitor are last administered, preferably within about 30 minutes after, and most preferably, being administered simultaneously with the CK2 inhibitor. Alternatively, “simultaneously” means that the medicant is administered within about 30 minutes before, and preferably within about 15 minutes before the CK2 inhibitor is first administered.

Physical disruption of the blood brain barrier includes by means of “mechanical” injury or other physical trauma that breaches the blood brain barrier in at least one location of the brain's vasculature. Chemical disruption includes by an agent that transiently permeabilizes the blood-brain barrier and allows the CK2 inhibitor to enter the brain from the blood stream via the brain microvasculature. Such permeabilizing agents are known, for example, bradykinin and bradykinin analogs, and activators of calcium-dependent or ATP-dependent potassium channels. (e.g., B. Malfroy-Camine, Method for increasing blood-brain harrier permeability by administering a hradykinin agonist of blood-brain barrier permeability, U.S. Pat. No. 5,112,596; J. W. Kozarich et al., Increasing blood brain harrier permeability with permeabilizer peptides, U.S. Pat. No. 5,268,164; Inamura, T. et al., Bradykinin selectively opens blood-tumor harrier in experimental brain tumors, J. Cereb. Blood Flow Metab. 14(5):862-70 [1994]; K. L. Black, Method for selective opening of abnormal brain tissue capillaries, U.S. Pat. Nos. 5,527,778 and 5,434,137; N. G. Rainov, Selective uptake of viral and monocrystalline particles delivered intra-arterially to experimental brain neoplasms, Hum. Gene. Ther. 6(12):1543-52 [1995]; N. G. Rainov et al., Long-term survival in a rodent brain tumor model by bradykinin-enhanced intra-arterial delivery of a therapeutic herpes simplex virus vector, Cancer Gene Ther. 5(3):158-62 [1998]; F. H. Barnett et al., Selective delivery of herpes virus vectors to experimental brain tumors using RMP-7, Cancer Gene Ther. 6(1):14-20 [1999]; WO 01/54771 A2; and WO 01/54680 A2).

Other embodiments of the inventive method are directed to the treatment of proliferative retinopathies, such as proliferative diabetic retinopathy, retinopathy of prematurity (retinopapillitis of premature infants treated with high concentrations of oxygen gas), proliferative vitreoretinopathy, or choroidal neovascularization associated with age-related macular degeneration. In treating such retinopathies, administration of the pharmaceutically acceptable composition can be local, such as by intravitreal or stereotactic injection of an aqueous solution or suspension, formulated to be compatible with the intraocular environment. Alternatively, local adminstration can be by way of eye drops, eye ointments or creams, or by a trans-eyelid patch. Alternatively, the pharmaceutically acceptable composition can be formulated as a contact lens or intraocular lens that contains and then releases the CK2 inhibitor to the eye. However, a systemic delivery route is also useful for delivering the CK2 inhibitor to the retinal tissue.

Another preferred embodiment of the inventive method is directed to treating proliferative glomerulonephritis, e.g., as frequently presents in patients with systemic lupus erythematosus. Here, proliferation of renal endothelial cells is inhibited by the inventive method. In this embodiment, administration of the pharmaceutically acceptable composition comprising the CK2 inhibitor is preferably by a systemic delivery route.

The present invention is also directed to an in vitro method of screening a potential antiangiogenic agent. Examples of agents that can be evaluated for potential antiangiogenic activity in accordance with the invention, include compounds or substances, whether or not these are newly known, isolated or synthesized; mixtures of compounds, such as cell, plant or animal extracts; or any combination of these. Culturing a plurality of mammalian endothelial cells is done by known cell culture techniques, typically by culturing in commercially available liquid aqueous cell culture medium in tissue culture flasks or multi-welled plates. Incubation is generally done at 37° C., in air containing 5% CO₂. In accordance with the in vitro method, the endothelial cells are cultured in the presence of signal molecules that induce proliferation of the cells. For purposes of the in vitro method, “signal molecules” are cytokines, growth factors, or hormones that can be introduced exogenously to induce or suppress a physiological response of the cells. Useful examples of signal molecules that induce proliferation of the cells include vascular endothelial growth factor (VEGF), placenta growth factor (PIGF), insulin-like growth factor (IGF)-I, platelet-derived growth factor (PDGF)-BB, epidermal growth factor (EGF), fibroblast growth factor (FGF)-2, and the like, e.g., interleukin, growth hormone (GH), interferon, hepatocyte growth factor (HGF), tumor necrosis factor(TNF)-α, and/or transforming growth factor (TGF)-α. Preferably, but not necessarily, combinations of different signal molecules are employed to yield a synergistic inductive effect, e.g., VEGF+IGF-I; VEGF+IGF-I+FGF-2+PIGF, or the like. Still in the presence of the signal molecules, a first population of the induced mammalian endothelial cells is exposed to the potential antiangiogenic agent. An amount of CK2 inhibitor sufficient to inhibit proliferation of the endothelial cells is provided by a culture medium containing a concentration of preferably about 10 μM to about 150 μM CK2 inhibitor, and more preferably about 25 μM to about 100 μM. Appropriate amounts of potential antiangiogenic agents vary and are determined by routine screening.

Detecting an antiangiogenic effect in the first population of endothelial cells is accomplished by one or more of any suitable assay means, such as detecting any effects on cellular proliferation, survival migration, and/or sprouting of endothelial cells: e.g., cell numbers, in vitro assay of capillary-like tube formation, or secondary sprouting, typically on or in various simulated extracellular matrix environments (e.g., BD or GFR Matrigel™).

As a positive control, separately from the first population, a second population of the mammalian endothelial cells cultured with the signal molecules is exposed to a selective inhibitor of protein kinase CK2 enzymatic activity, as described herein, in an amount sufficient to inhibit proliferation of the endothelial cells in the presence of the signal molecules and in the absence of the potential antiangiogenic agent. Detection of an inhibitory effect on cellular proliferation in the second population is by the same detection mean(s) employed with respect to the first population. The results from the first and second populations are compared, and an inhibitory effect in the first population similar to the inhibitory effect in the second population indicates an antiangiogenic property of the potential antiangiogenic agent.

Appropriate additional controls for use in the in vitro screening method will be self-evident to the skilled artisan. Such controls can include: (1) a population of endothelial cells administered sterile aqueous culture medium (or appropriate vehicle) alone in the presence of the signal molecules; (2) a population receiving the potential antiangiogenic agent in the absence of the signal molecules; and/or (3) a population receiving the CK2 inhibitor in the absence of the signal molecules.

The present invention is also directed to a kit for the treatment of a disease by inhibiting angiogenesis. The kit is useful for practicing the inventive methods. The kit is an assemblage of materials or components, including the pharmaceutically acceptable composition comprising at least one CK2 inhibitor, as described above.

Instructions for using the CK2 inhibitor in the inventive methods are also included in the kit. “Instructions for use” typically include a tangible expression describing the reagent concentration or at least one treatment method parameter, such as the relative amounts of reagents to be admixed, maintenance time periods for reagent admixtures, temperature, buffer conditions, administration method, dose, or dosing frequency, or the like, typically for an intended purpose.

Optionally, the kit also contains other useful components, such as, diluents, buffers, pharmaceutically acceptable carriers, syringes, stents, catheters, or pipetting or measuring tools.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures.

The components are typically contained in suitable packaging material(s). As employed herein, the phrase “packaging material” refers to one or more physical structures used to house the contents of the kit. The packaging material is constructed by well known methods, preferably to provide a sterile, contaminant-free environment.

The packaging materials employed in the kit are those customarily utilized in pharmaceutical systems. As used herein, the term “package” refers to a suitable solid matrix or material such as glass, plastic, paper, cardboard, foil, and the like, capable of holding the individual kit components. Thus, for example, a package can be a glass vial used to contain suitable quantities of the CK2 inhibitors. The packaging material generally has an external label which indicates the contents and/or purpose of the kit and/or its components.

As used herein, the term “mammal” or “mammalian” refers to vertebrate animals belonging to the class Mammalia, including all that possess hair and suckle their young, e.g., humans, non-human primates (e.g., monkeys, baboons, apes), rodents (e.g., rats, mice, guinea pigs), lagomorphs (e.g., rabbits), bovine, porcine, ovine, canine, feline, equine, elephant, and the like.

A “tissue” is a group of similar cells united to perform a specific physiologic function. For example, vascular tissue is found throughout the body to carry blood; and blood itself is regarded as a tissue, such that a blood sample is also a tissue sample for purposes of the present invention. The tissue can be organized as an organ, for example, an eye, kidney (e.g., renal tissue), liver, heart, brain, esophagus, stomach, intestine, pancreas, breast, ovary, uterus (e.g., uterine tissue), testis, prostate, spleen, parotid gland, adrenal, submaxillary gland, sublingual gland, lymph node, lung, bone marrow, mediastinum, or skin, or as a subpart of an organ, such as retinal tissue, choroidal tissue, vascular tissue, cervix uteri, or endometrial tissue. For purposes of the present invention, a malignant tumor is also a tissue, i.e., a “malignant tissue.” Optionally, a tissue sample can be obtained by being collected from a mammalian subject by direct sampling, or by being gathered, received and/or transported for the purpose of practicing the method. Direct sampling of tissue is by any known means, including but not limited to, blood draw or biopsy by any suitable surgical technique, such as laproscopic biopsy, percutaneous biopsy, stereotactic biopsy, tissue swab or scrape, and the like. A tissue sample can alternatively be obtained from cultured mammalian cells originating from a primary tissue sample. Tissue samples can optionally be stored by well known storage means that will preserve the cells in a viable condition, such as quick freezing, or a controlled freezing regime, in the presence of a cryoprotectant, for example, dimethyl sulfoxide (DMSO), glycerol, or propanediol-sucrose.

“Endothelium” is a layer of epithelial cells that lines the cavities of the heart, blood vessels, lymph vessels, retina, and the serous cavities of the mammalian body, originating from the mesoderm. Endothelial cells constituting the endothelium can come from either existing endothelium or from bone marrow-derived endothelial precursor cells circulating in the blood.

An “endothelial cell” is a typically thin, flattened cell that is a constituent cell of the endothelium, is part of an endothelial tissue sample, or is a cultured cell originating from an endothelial tissue sample. A vascular endothelial cell is an example. The expressions “differentiated endothelial cell” or “mature endothelial cell” are used herein interchangeably, and denote endothelial cells expressing physiological and/or immunological features of terminally differentiated endothelial cells, including markers, such as CD31, CD36 and CD62, V-Cadherin. (Reyes M., et al. Origin of endothelial progenitors in human postnatal bone marrow. J Clin Invest. 2002. 109(3):337-346). Included among endothelial cells are secondary, tertiary, and further cultured cells derived from a primary endothelial cell culture, in vitro, which cells continue to exhibit surface markers known to be characteristic of endothelial cells.

An “endothelial precursor cell” (EPC) is a stem cell that can differentiate into a mature endothelial cell in response to certain cytokines. Endothelial precursor cells characteristically express AC133, CD 166, AML-1, uPA, tPA, CD31, flk-1, flt-1, tie-2, the capacity to take up acetylated LDL, and the presence of cytoplasmic Weibel-Palade bodies, in contrast to hematopoietic precursor cells that develop from a stem cell lineage in common with endothelial precursor cells. (See, e.g., Choi, K. et al., A common precursor for hematopoietic and endothelial cells, Development 125:725-32 [1998]). EPCs characteristicly overexpress telomerase, compared to mature endothelial cells. Morphologically EPCs are polymorphic; they can be flattened, spherical, or can possess a sprout morphology that exhibits one or more morphological processes about 50 to about 500 micrometers long extending from the central mass of the cell.

While the invention has been described with reference to its preferred embodiments, it will be appreciated by those skilled in this art that variations may be made departing from the precise examples of the methods and compositions disclosed herein, which, nonetheless, embody the invention defined by the following claims.

### EXAMPLES

### Example 1

### Synergistic Effects of Angiogenic Growth Factors on Cultured Retinal Endothelial Cells (REC)

Alterations of angiogenic growth factors and retinal basement membranes (BMs) are important for diabetic retinopathy (DR) pathogenesis. Consequently, whether angiogenic growth factors can mediate angiogenic behavior of retinal endothelial cells (REC) in an additive manner was examined.

Human REC (from normal, diabetic and patients with DR [“DR REC”]) and bovine REC were cultured in monolayer (for migration assay) or on top of Matrigel™ where cells form capillary-like tubes. They were treated with angiogenic growth factors or their combinations (at 10 ng/ml of each factor), and seeded with or without TN-C at 10-50 μg/mL. Cell numbers were determined by MTS assay (Promega Corp., Madison, Wis.). Tube length and number, and cell migration were assessed microscopically.

Retinal endothelial cells were isolated from fresh bovine eyes (Sierra for Medical Science, Santa Fe Springs, Calif.) using a modification of the method of Grant and Guay. (Grant and Guay, Plasminogen activator production by human retinal endothelial cells of nondiabetic and diabetic origin, Invest. Ophthalmol. Vis. Sci. 32, 53-64 [1991]). For some experiments, human REC were cultured from healthy and diabetic donor eyes obtained from the National Disease Research Interchange (NDRI, Philadelphia, Pa.). NDRI has a human tissue collection protocol approved by a managerial committee and subject to National Institutes of Health oversight. Briefly, aseptically dissected retinas were manually triturated and passed through a sterile 45 μm nylon mesh (Tetko Inc./Sefar America Inc., New York, N.Y.) followed by extensive rinsing with dissecting buffer [50% fetal calf serum (Omega Scientific Inc., Tarzana, Calif.) in Dulbecco's PBS (Invitrogen/Life Technologies, Carlsbad, Calif.)]. The pooled retentate was digested with collagenase (Worthington Biochemical Corp., Lakewood, N.J.) in Ca⁺⁺/Mg⁺⁺-free PBS (Invitrogen) with moderate stirring for ˜30 min. The digest was resuspended in incomplete REC medium [50% F-12, 50% low-glucose DMEM with antibiotics/antimycotics (Invitrogen) and 10% fetal calf serum (FCS)] and centrifuged at 400×g for 5 min. The pellet was resuspended in high serum, complete, BREC medium [same as incomplete medium plus ITS (insulin/transferrin/selenium), ECGS (endothelial cell growth supplement), all from Sigma-Aldrich Co., St. Louis, Mo., and 20% FCS]. After the first passage cells were routinely cultured in complete BREC medium with 10% FCS (growth medium). Only passages 3-7 were used for experiments. Cultures were often checked for purity by immunostaining with a rabbit polyclonal antibody against von Willebrand factor (Sigma-Aldrich).

In vitro Matrigel™ assay of capillary-like tube formation and secondary sprouting. Matrigel™, a tumor extract containing major basement membrane components (10 mg/mL protein) is obtained from Collaborative Research, which preparation was found to be superior over three other brands in terms of tube formation. Briefly, 50 μL of reconstituted basement membrane matrix from mouse EHS tumor (BD or GFR Matrigel™; Becton Dickinson Labware, Bedford, Mass.) were dispensed with frozen pipettes into each well of a previously frozen, sterile 96-well plate sitting on wet ice and allowed to solidify for 1 hr at room temperature or 37° C. Approximately 5×10⁴ or 7.5×10⁴ REC in a 100 μL volume were seeded into each triplicate well. Human recombinant or purified growth factors were added to a final concentration of 10 ng/mL (or as noted) in 0.5% FCS incomplete BREC medium. Capillary-like tube structures formed by REC on reconstituted basement membrane matrix were photographed at various intervals ranging from 12-72 hr; pictures were scanned, digitized and analyzed using image-processing software. For the secondary sprouting assay, cells on reconstituted basement membrane matrix (BD Matrigel™) were seeded as above but incubated in 0.5% FCS incomplete BREC medium without any growth factors for 3 days, allowing for tube formation and collapse. On day 3, human recombinant or purified growth factors were added to a final concentration of 10 ng/mL (or as noted) in low-serum incomplete BREC medium and incubated for another 5-6 days. Digital photographs were obtained with a Kodak MDS 100 camera attached to a Leitz DM IL inverted microscope. Digitized images obtained with a Kodak MDS 100 video camera were stored on compact discs and quantified with NIH Image 1.62 software. The number of living cells in the sprouting colonies were determined using the MTS cell proliferation assay.

The cells form tubes on Matrigel™ by 16 hr, and by 48 hr, without TN-C or growth factors, the tubes collapse. Secondary sprouting with Matrigel™ invasion starts by day four in culture. Cultures were monitored microscopically. Culture medium with or without growth factors or inhibitors was changed every two-three days.

Migration assay. REC migration rates were examined in a wound healing assay, where cells migrate over time into the scrape wound in a monolayer. Briefly, cells were seeded in 24-well plates and allowed to reach confluence in growth medium. Prior to growth factor treatment, cells were serum-starved overnight in incomplete BREC medium with 0.5% FCS. All monolayers within an experiment were wounded with a single sterile wood stick of constant diameter, to ensure uniformity in the wound areas among different treatments. Wounded monolayers were then rinsed with low-serum medium to remove detached cells and treated with various combinations of human growth factors at 10 ng/mL each. On day 7, cells were rinsed 3× with PBS and fixed with methanol for 15 min, rehydrated with dH₂O and stained with Meyer's hematoxylin for 5 min, followed by destaining with dH₂O. All wells were photographed with a 4× or 10× objective using a Kodak MDS 100 digital camera attached to a Leitz DM IL inverted microscope. The original wound area was measured at 0 hr and used as a baseline for comparison to the treated wells at the conclusion of the experiment. The number of cells migrated into the wound was determined. Migrating cell counting was automated using the AAB (Advanced American Biotechnology, Fullerton, Calif.) software. Data were calculated and statistically analyzed (Spirin K S et al., Basement membrane and growth factor gene expression in normal and diabetic human retinas, Curr. Eye Res. 18:490-499 [1999]) relative to control cultures that received the same concentrations of bovine serum albumin instead of growth factors and/or inhibitor, compared to vehicle instead of inhibitor after wounding. Inhibitors were added 30 min before growth factors.

REC proliferation and survival assays. 96-well plates were coated with various amounts of TN-C or vehicle. 5×10³ cells were added to each triplicate well in low-serum REC medium with various amounts of growth factors (0.5% FCS incomplete BREC medium containing 10 ng/ml of human IGF-I, FGF-2, VEGF, PlGF and PDGF-BB [R&D Systems Inc., Minneapolis, Minn.]). Cell numbers were determined on days 4-7 using the MTS cell proliferation assay (Promega Corp.) according to manufacturer's instructions. Survival was measured in the same way using high glucose (30 mM) or chemical hypoxia (2 mM sodium azide) or serum-free medium to induce cell death. Cell numbers were determined on days 4-7 using MTS assay (Promega).

Immunohistochemistry. Secondary sprouting colonies were scooped out of the reconstituted basement membrane matrix, washed with Dulbecco's PBS (Invitrogen) and embedded in OCT (Ted Pella Inc., Redding, Calif.). Blocks were frozen and cryosectioned. Some slides were stained with hematoxylin and eosin using standard protocols in order to locate the sprouting colonies within pieces of matrix. Unfixed 5-μm sections were double stained with a rabbit polyclonal antibody against von Willebrand factor (Sigma) and a rat monoclonal antibody against the laminin γ1 chain, clone A5 (Ljubimov et al, Distribution of individual components of basement membrane in human colon polyps and adenocarcinomas as revealed by monoclonal antibodies, Int. J. Cancer 50:562-6[1992]), at 20 μg/mL for one hour at room temperature. Slides were washed extensively with PBS and incubated for another hour with a 1:80 dilution of their respective cross-species preabsorbed secondary antibodies (Chemicon International, Temecula, Calif.) coupled to fluorescein or rhodamine. After extensive washing, slides were mounted in 50% glycerol in PBS and photographed using an Olympus BH-2 fluorescent microscope.

Image and Statistical Analysis. All the treatment data sets were individually compared to their respective controls (unless otherwise specified) by the paired Student's t-test using the GraphPad Prism 3.0 program (GraphPad Software, San Diego, Calif.). In some experiments, one treatment was compared to several others using a non-parametric one-way ANOVA test (GraphPad Software). Tube formation images were processed by background subtraction, thresholding and measurement of total length of tubes using Adobe Photoshop v5.0 (Adobe Systems Inc., San Jose, Calif.) and the Image Processing Toolkit v3.0 (Reindeer Games, Inc., Gainesville, Fla.).

Treatment of REC cultures. Duplicate REC cultures on plastic (for migration) or Matrigel™ with the same number of cells per dish are treated with previously established working concentrations of signaling inhibitors and/or select growth factor combinations. Treatments begin at the time of seeding the cells and medium is changed every other day. Single growth factors are used as negative controls since their modulation of TN-C effects was minimal. Working growth factor concentrations were as follows: VEGF, 1-50 ng/mL depending on the assay; PlGF, 100 ng/mL; FGF-2, 10-100 ng/mL; IGF-I, 25-100 ng/mL; PDGF-BB, 10-100 ng/mL. When used as combinations, each growth factor was supplied at 10 ng/mL for optimum synergy. The already tested inhibitors of signaling molecules (Sigma, Calbiochem, BIOMOL) were used at the following optimized doses: protein kinase A (inhibitor: H89 [25 μM]), PKC (inhibitor: calphostin C [2.5 μM]), PKC-β (inhibitor: LY379196 [50 nM]), Ca²⁺/calmodulin kinase II (inhibitor: KN-93 [0.5 μM]), CK1 (inhibitor: CKI-7 [50 μM]), MEK-ERK (inhibitor: PD98059 [10 μM]), p38 MAP kinase (inhibitor: SB202190 [10 μM]), PI3 kinase (inhibitor: wortmannin [100 nM]), CK2 (inhibitors: emodin [20-25 μM] and DRB [20-25 μM]), CK2 and other kinases (inhibitor: quercetin [50 μM]); and a negative control for kinase inhibition (SB202474 [10 μM]). In some experiments cells were grown in hyperglycemic medium with 30 mM glucose.

### Example 2

### Synergistic Effects of Growth Factors on Angiogenic Cellular Behaviors

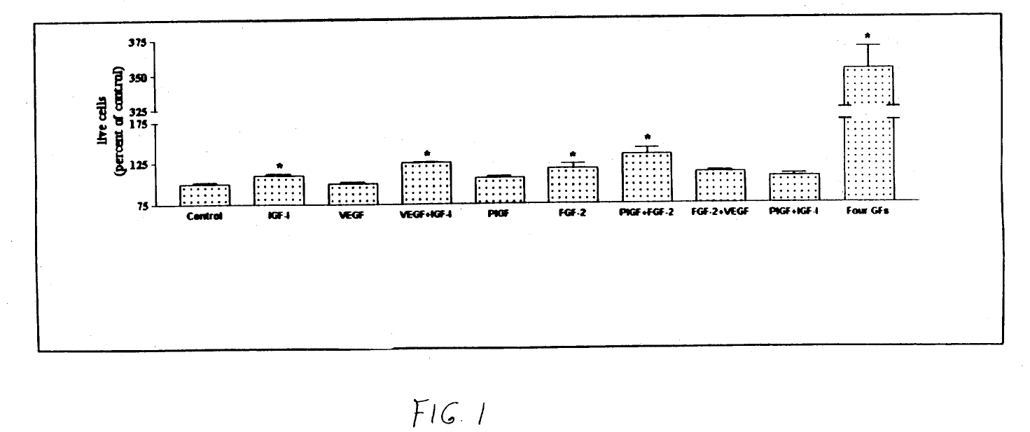

It was found that growth factors synergized to promote REC angiogenic behavior. Growth factor activities were rather selective. IGF-I preferentially synergized with VEGF, but FGF-2 coupled with PIGF (FIG. 1). PDGF-BB had a slight preference for FGF-2. However, it was so potent in REC by itself, that other factors only mildly enhanced its action. Individual growth factor effects on REC behavior varied. For example, VEGF had little effect on REC survival but significantly enhanced migration. IGF-I was the opposite. However, VEGF+IGF-I exerted an additive effect on cell survival, tube formation, sprouting, migration, and proliferation. REC treated with combinations of four or five growth factors showed significant, several-fold, enhancement of most angiogenic parameters tested (FIG. 1). Therefore, some angiogenic responses may be triggered only by growth factor combinations.

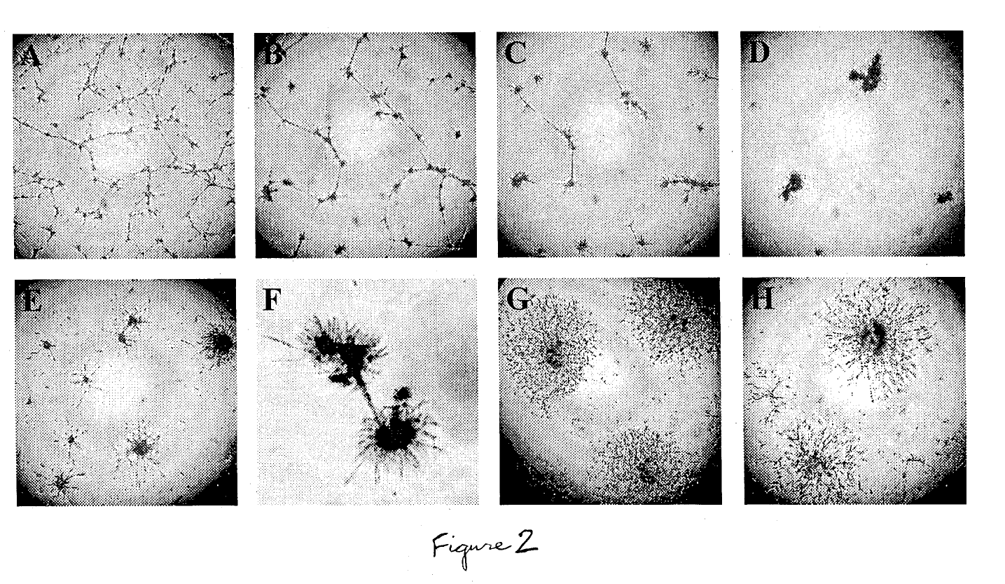

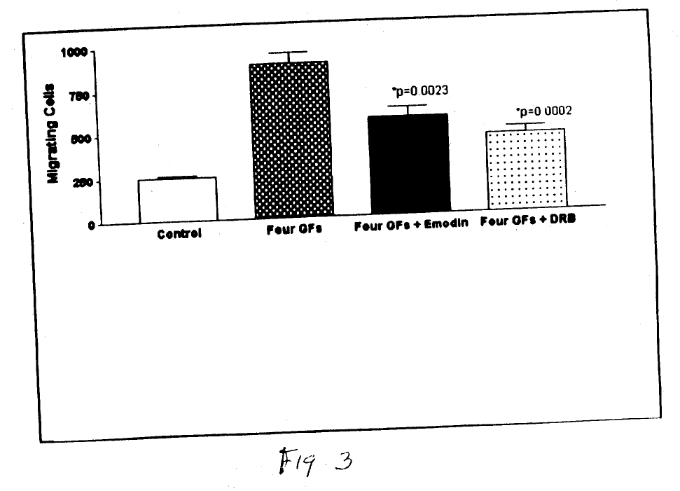

Vascular damage in DR is followed by angiogenic burst that creates a network of leaky and fragile vessels. We detected a similar process in REC cultured on Matrigel™. It was known that endothelial cells plated on this matrix stopped proliferating, formed capillary-like hollow tubes for 24-48 hr did not invade the matrix, collapsed into clumps, and died (Benelli R, Albini A, In vitro models of angiogenesis: the use of Matrigel, Int. J. Biol. Markers 14:243-246 [1999]). This was thought to be the endpoint of the assay. However, we observed that some cells survive following tube collapse. They 1) proliferate, 2) migrate, 3) form spherical colonies that remain alive for weeks, 4) invade basement membrane matrix (Matrigel™), and 5) can reassemble into larger tubes. We call this novel phenomenon “secondary sprouting” (FIG. 2). Angiogenic factors can enhance and modulate this process. Secondary sprouting was stimulated on average three-fold by PDGF-BB and four-fold by FGF-2, whereas other individual factors were less potent. Growth factor combinations were again more effective than individual factors: VEGF+IGF-I, 150%, PIGF+FGF-2, five-fold, and all these four factors, up to six-fold. Basal and growth factor-enhanced secondary sprouting could be decreased by inhibitors of CK2 but not of several other key signaling molecules. We also observed that DM and DR REC exhibited a higher sprouting ability than normal REC.

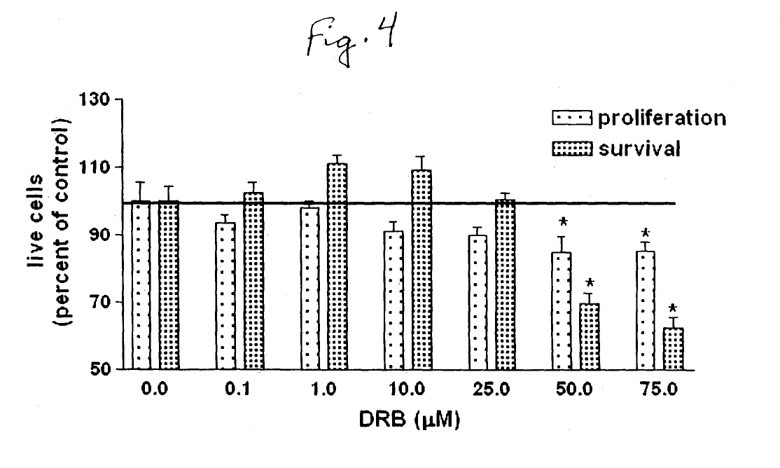

FIG. 4 shows the effect of DRB on bovine REC proliferation and survival. Cells were plated in medium with 0.5% (survival) or 10% serum (proliferation) containing various concentrations of DRB. The number of live cells was measured on day 6 with MTS assay. Bars represent mean±SDEM of two individual experiments in triplicate. The results show that DRB significantly lowers cell number at both serum concentrations.

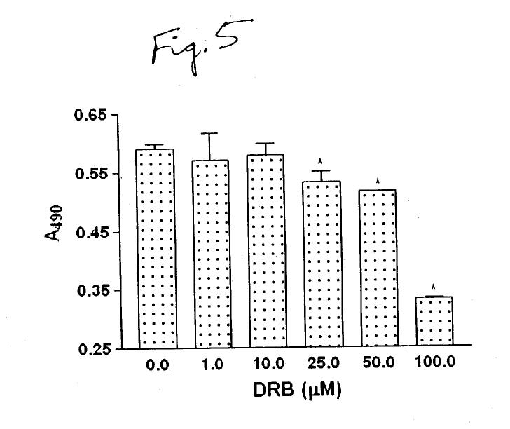

FIG. 5 shows the effect of DRB on bovine REC secondary sprouting. Cells were seeded on Matrigel™ in medium with 0.5% serum containing various concentrations of DRB. The number of live cells was measured on day 9 with MTS assay. Bars represent mean±SDEM of two individual experiments in duplicate. The results show that DRB significantly decreases cell number starting at 25 μM.

### Example 3

### Gene Array Analysis of Growth Factor Action on Normal REC and DR REC

Normal REC and DR REC gene expression patterns were compared by gene array analysis. Normal, diabetic and DR autopsy human eyes are obtained from National Disease Research Interchange (NDRI), within 24 hours after death. These eyes are used to isolate REC for culture as described hereinabove. Cultures can be used up to the fourth passage, and viable cultures can be cryogenically stored. Cultures of normal, diabetic and DR REC are established from autopsy human eyes and routinely checked for purity using von Willebrand factor immunostaining as described hereinabove. Cells are cultured in 50% F-12, 50% low-glucose DMEM with antibiotics/antimycotics (GIBCO/BRL), insulin-transferrin-selenite, ECGS (Sigma Chemical Co.), and 20% FCS. Statistical analysis of results is done with GraphPad Prism software (GraphPad Software).

In experiments, normal, diabetic, and DR REC were grown for seven days with or without 10 ng/ml VEGF, or 10 ng/mL IGF-I, or 10 ng/mL each VEGF and IGF-I, in medium with 0.5% serum. Long-term rather than short-term treatment was chosen because diabetes develops over a considerable time period. RNA isolated from REC was reverse-transcribed using Smart™ cDNA synthesis method (Clontech), to produce full-length cDNA. Two normal cases or two DR cases were pooled together. This cDNA was PCR-amplified with a short number of cycles and used as a probe for Clontech Atlas Human 1.2 1,200-gene arrays, according to the manufacturer's instructions. This technique had previously been refined and verified by Northern analysis and fully correlated gene array data with protein expression. (Spirin K S et al., Analysis of gene expression in human bullous keratopathy corneas containing limiting amounts of RNA, Invest. Ophthalmol. Vis. Sci. 40:3108-3115 [1999]). Samples were normalized to several housekeeping genes and the analysis was done with available AtlasImage 2.0 software (Clontech). Signal ratio>2 between samples was considered significant as per manufacturer's recommendation.

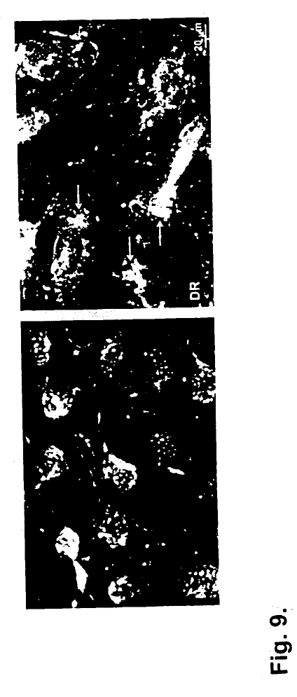

The gene expression pattern of untreated DR REC showed relatively increased expression of pro-apoptotic genes (Table 1), in agreement with known apoptosis activation in diabetic retinas (Gerhardinger C et al., IGF-I mRNA and signaling in the diabetic retina, Diabetes 50:175-183 [2001]). These included caspases, Fas antigen and ligand, tumor necrosis factor (TNF)-α and its receptors, and bcl-2 killer (BAK). Expression of mRNAs of VCAM-1 and its α₄ integrin receptor, related to the activated endothelium, were also elevated. However, some proliferation-related genes (STAT3, c-jun and c-fos protooncogenes, G1/S cyclin E, transcription factors E2F, ets-1, NF-κB, intermediary factor 1β) were also increased compared to normal cells. DR-upregulated ets-1 and NF-κB, which can induce TN-C expression that increases in DR retinas. (E.g., Jones F S, Jones P L, The tenascin family of ECM glycoproteins: structure, function, and regulation during embryonic development and tissue remodeling, Dev. Dyn. 218:235-2597 [2000]; Spirin K S et al., Basement membrane and growth factor gene expression in normal and diabetic human retinas, Curr. Eye Res. 1999;18:490-499 [1999]). DR REC had increased CK2 and its binding protein, protein phosphatase 2 (PP2A), consistent with a significant role for CK2 in DR development. FIG. 9 shows CK2 α subunit expression in cultured REC of normal (N) and diabetic retinopathic (DR) origin as detected by immunohistochemistry. In normal cells, a comparatively weak nuclear staining is mostly seen. In DR cells, there was also distinct cytoplasmic staining (arrows). The staining intensity was higher in DR cells, indicating overexpression of CK2. Cathepsins decreased in DR REC, in line with previously observed reduced basement membrane proteolysis in DR retinas. (Grant M B et al., Plasminogen activator inhibitor (PAI)-1 overexpression in retinal microvessels of PAI-1 transgenic mice, Invest. Ophthalmol. Vis. Sci. 41:2296-2302 [2000]).

Growth factor treatment mostly caused coordinate gene expression changes in normal and DR REC (Table 1). A minority of genes were changed selectively, either in normal or DR cells, and VEGF-treated cells did not display an increase of pro-apoptotic genes (not shown). Certain proliferation-relited genes were upregulated by VEGF, including transcription factor Sp2, elongation factors SII and SIII, and signaling molecules, S6 kinase and JAK1. VEGF downregulated various phosphatases in normal and DR REC suggesting activation of phosphorylation-dependent metabolic pathways, while exposure to IGF-I alone caused a decrease of pro-apoptotic genes (not shown). A combination VEGF+IGF-I caused a dramatic downregulation of pro-apoptotic genes (activated in DR) and an increase of proliferation-related genes (data not shown). Particularly, a group of several stress-related MAP kinases associated with endothelial and pancreatic β-cell apoptosis in diabetes (Davis R J, Signal transduction by the JNK group of MAP kinases, Cell 103:239-252 [2000]) was also downregulated. At the same time, key signaling molecules, PLCγ2, PI3 kinase α, and ras p120 activator were increased by VEGF+IGF-I.

Gene expression profile of DR REC showed increases of many apoptosis-associated genes (Table 1). CK2 gene expression was elevated in DR cells. Synergistic action of angiogenic growth factors on normal and DR REC gene expression was consistent with other data from cell migration, proliferation, and secondaryh sprouting (on Matrigel™) assays. Moreover, the gene expression data in REC (Table 1) closely parallel the results obtained by other methods not related to gene expression analysis. (Davis R J, Signal transduction by the JNK group of MAP kinases, Cell 103:239-252 [2000]; Franklin R A, McCubrey J A, Kinases: positive and negative regulators of apoptosis, Leukemia 14:2019-2034 [2000]). Therefore, there is strong reason to believe that the major changes detected by gene arrays translate into gene product changes, significantly including increased CK2 expression in DR REC.

TABLE 1
Gene array analysis of gene expression in human
REC cultures from patients with diabetic retinopathy
(DR) compared to normal humans.

| Increased Expression in | Decreased Expression in |
|---|---|
| DR vs. Normal | DR vs. Normal |
| E2F transcription factor 3 | Autocrine motility factor |
| | receptor |
| Gl/S cyclin E | BMP1 |
| Caspase 3 | EGF |
| Caspase 4 | PAI-1 |
| Caspase 6 | Integrin β3 |
| BCL2 killer (BAK) | BCL2-like 2 |
| Thrombopoietin receptor | MAPK7 |
| precursor | |
| Protein-tyrosine phospha- | Cadherin 14 (M-cadherin) |
| tase 1E | |
| Protein phosphatase 2A | Fas-activated ser/thr |
| | kinase |
| Ras-related protein | IL-5 |
| RAP-1A/KREV-1 | |
| N-myc protooncogene | L-myc protooncogene |
| Transcription intermediary | Cathepsin D |
| factor 1β | |
| CK2 α | Cathepsin C |
| STAT3 | STAT1 |
| IGF-II | Integrin β5 |
| VEGFR3 | |
| FAS antigen | |
| FAS ligand | |
| TNF-α | |
| TNFR superfamily member 1B | |
| TNFR superfamily member 1A | |
| Cell division control | |
| protein 2 (CDC2) | |
| c-kit protooncogene | |
| fos-related antigen 2 | |
| jun-D protooncogene | |
| c-jun protooncogene | |
| ets-1 p54 | |
| ets-related gene transforming | |
| protein | |
| Microtubule affinity-regulating | |
| kinase 3 | |
| Ephrin-B receptor 2 (EPH-3) | |
| VCAM-1 | |
| Integrin α4 | |
| NF-κB | |
| EGFR substrate | |
| MAPK3 | |

### Example 4

### CK2 Involvement in REC Behavior, Growth Factor Action, and Retinal Neovascularization

Methods. In order to facilitate the optimization of CK2 inhibitor doses and the application of various assays, large numbers of bovine REC were employed in some experiments. The bovine REC were very similar to human REC in all assays and in their responses to growth factors. Cultured cells were treated with growth factor combinations with or without inhibitors of the following molecules: protein kinase A (inhibitor: H89), PKC (inhibitor: calphostin C), PKC-β (inhibitor: LY379196), Ca²⁺/calmodulin kinase II (inhibitor: KN-93), CK1 (inhibitor: CKI-7), MEK-ERK (inhibitor: PD98059), p38 MAP kinase (inhibitor: SB202190), PI3 kinase (inhibitor: wortmannin), CK2 (selective inhibitors: emodin and DRB), CK2 and other kinases (inhibitor: quercetin).

Results. Preliminary results had demonstrated that H-7, a broad-spectrum protein kinase inhibitor, stabilized REC tubes on Matrigel™, inhibited secondary sprouting, migration and proliferation (not shown). Consequently, attempts were made to identify specific kinases that were inhibited by H-7 and played a role in these events. Most inhibitors tested caused minor to moderate effects in all assays. However, inhibitors that could block CK2 (quercetin, emodin DRB) potently inhibited basal and growth factor-stimulated proliferation, secondary sprouting, migration, and tube formation (FIG. 5). As emodin and DRB are specific CK2 inhibitors, and other inhibitors had only a slight effect, the observed inhibition by quercetin was most probably due to blocking CK2 activity. Actinomycin D caused only minor changes in angiogenic assays, implying that CK2 effects on REC did not involve its known impact on transcription (e.g., Guerra B, Issinger O G, Protein kinase CK2 and its role in cellular proliferation, development and pathology, Electrophoresis 20:391-408 [1999]).

A specific protein kinase CK2 inhibitor, emodin, was tested for its ability to inhibit neovascularization in oxygen-induced retinopathy in newborn mice (7-day old C57BL/6J mouse pups weighing 4-5 g each) in a previously described animal model. (Smith, L E et al., Invest Ophthalmol Vis Sci 35: 101-111 [1994]; Rotschild, T et al., Pediatr Res 1999;46: 94-100). Briefly, these experiments were done as described [Mino R P et al, Adenosine A2B antagonists reduce retinal neovascularization, Curr. Eye Res. 2001, In press.]. Wild type C57BL/6J mice (Jackson Laboratory) were used. The retinopathy group was placed in 75% oxygen at postnatal day seven and maintained in these conditions with their nursing mothers for five days. These mice were then returned to normal air and maintained for another five days. Normoxic control mice are maintained in normal air for the same duration as test mice and under the same conditions of light cycle and temperature. Mice were anesthetized with Ketamine-Xylazine (in a ratio 0.1:0.1:0.5 with PBS injected at 5 μL/g body weight) and perfused through the left ventricle with 4% paraformaldehyde in 0.1 M phosphate buffer pH 7.4 with 50 mg/mL 2×10⁶ Da fluorescein-dextran (Sigma Chemical Co.). The eyes were enucleated and fixed in 4% paraformaldehyde for 18 h. The sclera and retinal pigment epithelium were stripped off the outer surface of the eye with jewelers forceps. The retina was dissected free of the lens and cornea, peripheral retinas are cut in five places and are flat-mounted with glycerol-gelatin. The retinas were viewed by fluorescence microscopy and photographed.

A minimum of 5 mouse pups were used per group. Emodin at 20-30 μg/g of body mass (or 10 μl/g) was prepared as a solution (70% ethanol) or suspension (PEG-Tween) in vehicle. Vehicle (“emodin solvent”) was 70% ethanol in initial experiments. In three later experiments, phosphate-buffered saline with 20% polyethylene glycol 400 (PEG 400)+2% Tween-80, pH 7.2, was used as vehicle. The PEG-Tween did not ensure emodin solubility, so the final mixture was a suspension that was sonicated briefly before injections. The latter vehicle proved to be better than 70% ethanol in terms of mouse survival.

Each mouse pup received two intraperitoneal injections of CK2 inhibitor or vehicle control daily. Injections started on the final day of hyperoxia (day 11 after birth) and continued throughout the subsequent normoxic period until the last day of experiment (day 17 after birth). The mice were euthanized as described herein above and their eyes were analyzed quantitatively for the extent of retinal neovascularization by the following method. On the fifth day after return to normoxia, the eyes from perfused mice were fixed in 4% paraformaldehyde and were embedded in paraffin. Serial 1-μm sections of whole eyes were cut sagitally, with 10 μm between sections, through the cornea and parallel to the optic nerve. Ten sections were counted from each eye resulting in sampling thickness of 110 μm in each eye. Sections were stained with hematoxylin-eosin to visualize cell nuclei under light microscopy. Human counters blinded to the treatment identity counted all nuclei above the inner limiting membrane (ILM) in 10 sections per each eye. Neovascularization rate in TN-C null retinas is calculated as the fraction of total nuclei over total nuclei in wild type or heterozygous control. Sections with the optic nerve were excluded, since normal vessels emanating from the optic nerve, though distinguishable from neomicrovasculature extending into the vitreous, fulfill the counting criterion and would have increased the error. Vascular cell nuclei were considered to be associated with new vessels if found on the ILM vitreal side. Pericytes were not identified in the neovascular tufts and have not been documented in neovasculature. Nevertheless, pericytes or their precursors may have been included in some cell counts. Results were statistically analyzed with a two-tailed Student t test using GraphPad Prism software program (GraphPad).

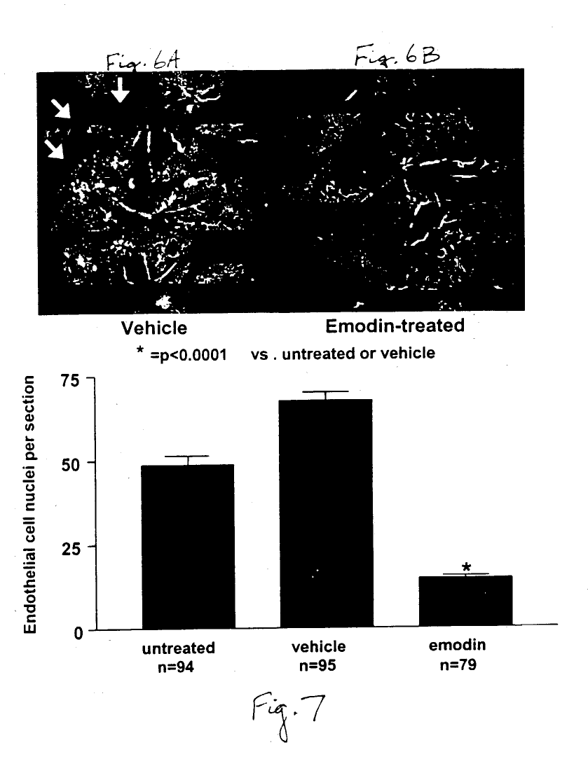

The FIG. 6 shows representative fluorescein angiograms of the retina from a vehicle-treated mouse (FIG. 6A) and of the retina from an emodin-treated mouse (FIG. 6B). There was significantly less vascularization in the emodin-treated mouse retina than in the vehicle control. Arrows show neovascular tufts prominent in the vehicle-treated animals. These tufts were much less pronounced in the emodin-treated pups (FIG. 6B) than the vehicle control group (FIG. 6A).

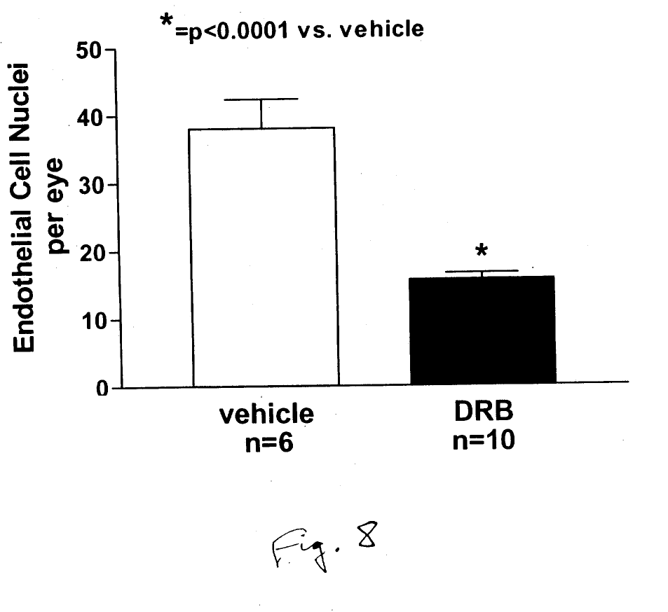

The FIG. 7 shows a quantitation of preretinal neovascularization in untreated, vehicle-treated and emodin-treated mouse retinas. Paraffin-embedded tissue sections were used and 3-4 sections per mouse eye were counted. Data represent mean of seven separate experiments, with a total of 24-30 mouse pups per group, (n=number of pups). Since the fellow eye (i.e., contralateral eye) of each mouse was stained by fluorescein, each embedded eye represents a separate animal. The results clearly show that emodin drastically diminished retinal neovascularization by 70-75%, with highly significant differences compared to either untreated or vehicle-treated animals (P<0.0001).

Another selective protein kinase CK2 inhibitor, DRB, was used in several experiments and the results were similar to those obtained with emodin, albeit the inhibition of retinal neovascularization was somewhat less pronounced. FIG. 8 shows a quantitation of preretinal neovascularization in untreated, vehicle-treated and DRB-treated mouse retinas. Data represent mean of two separate experiments, with a total of 3-5 mouse pups per group, (n=number of pups). Since the fellow eye (i.e., contralateral eye) of each mouse was stained by fluorescein, each embedded eye represents a separate animal. The results show that DRB diminished retinal neovascularization by about 60%, with highly significant differences compared to either untreated or vehicle-treated animals (P<0.0001).

Together, these data demonstrate that specific inhibitors of protein kinase CK2 are indeed capable of efficiently inhibiting retinal neovascularization in the oxygen-induced mouse retinopathy model. Since it is difficult to inject compounds in the mouse eye, injections were done intraperitoneally. Even with this route of administration, the beneficial effect in the retina was very pronounced. Previous work with a less selective inhibitor, quercetin, also showed substantial effect with intramuscular injections (data not shown). Importantly, the treatment reduced neovascular tufts in the retina, with little, if any effect on pre-existing retinal vasculature, or vasculature in other parts of the body.

We claim:

1. A method of inhibiting angiogenesis in a mammal, comprising:
administering to the mammal a pharmaceutically acceptable composition comprising a selective inhibitor of protein kinase CK2 enzymatic activity, such that an effective amount of the inhibitor is delivered to a tissue in the mammal, said tissue comprising endothelial cells, and protein kinase CK2 enzymatic activity is inhibited in a plurality of the cells, whereby an antiangiogenic effect in the tissue results.

2. The method of claim 1, wherein the inhibitor of protein kinase CK2 enzymatic activity is emodin or aloe-emodin.

3. The method of claim 1, wherein the inhibitor of protein kinase CK2 enzymatic activity is 5,6-dichloro-1-β-D-ribofuranosylbenzimidazole (DRB).

4. The method of claim 1, wherein the inhibitor of protein kinase CK2 enzymatic activity is 4,5,6,7-tetrabromobenzotriazole (TBB).

5. The method of claim 1, wherein the mammal is a human.

6. The method of claim 1, wherein the tissue is a malignant tissue.

7. The method of claim 1, wherein the tissue is retinal tissue.

8. The method of claim 1, wherein the tissue is choroidal tissue.

9. The method of claim 1, wherein the tissue is renal tissue.

10. The method of claim 1, wherein the tissue is uterine tissue.

11. The method of claim 1, wherein the endothelial cells are vascular endothelial cells.

12. Use of a selective inhibitor of protein kinase CK2 enzymatic activity in the manufacture of a medicament for inhibiting angiogenesis.

13. The use of claim 12, wherein the inhibitor of protein kinase CK2 enzymatic activity is emodin or aloe-emodin.

14. The use of claim 12, wherein the inhibitor of protein kinase CK2 enzymatic activity is 5,6-dichloro-1-β-D-ribofuranosylbenzimidazole (DRB).

15. The use of claim 12, wherein the inhibitor of protein kinase CK2 enzymatic activity is 4,5,6,7-tetrabromobenzotriazole (TBB).

16. An in vitro method of screening a potential antiangiogenic agent, comprising:
(a) culturing a plurality of mammalian endothelial cells in the presence of signal molecules that induce proliferation, survival, migration, and/or sprouting of the cells;
(b) then exposing a first population of the plurality of mammalian endothelial cells to the potential antiangiogenic agent, and detecting an effect on cellular proliferation, survival, migration, and/or sprouting in the first population;
(c) further, separately from the first population, exposing a second population of the plurality of mammalian endothelial cells to a selective inhibitor of protein kinase CK2 enzymatic activity, in an amount sufficient to inhibit proliferation, survival, migration, and/or sprouting of the endothelial cells, and detecting an inhibitory effect on cellular proliferation, survival, migration, and/or sprouting in the second population; and
(d) comparing the detected effect of the potential antiangiogenic agent on cellular proliferation, survival, migration, and/or sprouting in the first population with the detected inhibitory effect of the selective inhibitor of protein kinase CK2 enzymatic activity on cellular proliferation, survival, migration, and/or sprouting in the second population, wherein an inhibitory effect in the first population similar to the inhibitory effect in the second population indicates an antiangiogenic property of the potential antiangiogenic agent.

17. The method of claim 16, wherein the inhibitor of protein kinase CK2 enzymatic activity is emodin or aloe-emodin.

18. The method of claim 16, wherein the inhibitor of protein kinase CK2 enzymatic activity is 5,6-dichloro-1-β-D-ribofuranosylbenzimidazole (DRB).

19. The use of claim 16, wherein the inhibitor of protein kinase CK2 enzymatic activity is 4,5,6,7-tetrabromobenzotriazole (TBB).

20. A kit for the treatment of a disease by inhibiting angiogenesis, comprising:
a pharmaceutically acceptable composition comprising a selective inhibitor of protein kinase CK2 enzymatic activity; and
instructions for using the composition in practicing the method of claim 1.

21. The kit of claim 20, wherein the inhibitor of protein kinase CK2 enzymatic activity is selected from the group consisting of emodin, aloe-emodin, 5,6-dichloro-1-β-D-ribofuranosylbenzimidazole (DRB), and 4,5,6,7-tetrabromobenzotriazole (TBB).


SEQUENCE LISTING (1 sequence)

SEQ ID NO: 1 - protein, 12 aa, Artificial Sequence
  CK2-specific substrate peptide
RRRADDSDDDDD